(19) United States
(12) Patent Application Publication
(10) Pub. No.: US 2023/0412455 A1
(43) Pub. Date: Dec. 21, 2023

(54) SYSTEMS AND METHODS FOR CALIBRATING SENSORS OF INTERNET OF THINGS (IoT) SYSTEMS

(71) Applicant: MachineSense, LLC, Baltimore, MD (US)

(72) Inventors: Biplab PAL, Ellicott City, MD (US); Utpal MANNA, Kolkata (IN); Ratneswar ROYCHOWDHURY, Kolkata (IN)

(73) Assignee: MachineSense, LLC, Baltimore, MD (US)

(21) Appl. No.: 18/460,121

(22) Filed: Sep. 1, 2023

Related U.S. Application Data
(63) Continuation of application No. 17/168,995, filed on Feb. 5, 2021, now abandoned.
(60) Provisional application No. 62/970,450, filed on Feb. 5, 2020.

(51) Int. Cl. H04L 41/0823; H04L 41/08

(52) U.S. Cl. CPC H04L 41/0823; H04L 67/12; H04L 41/0879

(57) ABSTRACT

Systems for performing an automated, in situ calibration of one or more sensors of internet of things (IoT) systems include one or more emulators capable of generating calibration set points that are applied to the sensors during the calibration process. The systems also include one or more computing devices configured to store the data necessary for the calibrations. The computing devices are further configured to monitor the sensor outputs during normal operation of the IoT systems to check for a loss of calibration or compromised data integrity; execute an automated calibration of a upon the detection of a loss of calibration or data-integrity issue; and validate the calibration results.

### CROSS-REFERENCE TO RELATED APPLICATIONS

This application is a continuation of U.S. application Ser. No. 17/168,995, filed Feb. 5, 2021. The '995 application clams the benefit under 35 U.S.C. 119(e) of U.S. provisional application No. 62/970,450, filed Feb. 5, 2020. The contents of each application are incorporated by reference herein in their entirety.

### INTRODUCTION

The use of sensor-based Internet of Things (IoT) systems is increasing rapidly. It is estimated that more than five billion IoT-enabled devices, of which approximately one billion are sensors, currently are deployed in such systems. The integrity of the data generated by these sensors, in general, is critical to the proper operation of the systems into which the sensors are incorporated.

Sensors typically require periodic calibration, and without such calibration, the output of a sensor may not be reliable. Also, many sensors tend to lose accuracy over time due to poor maintenance and harsh environmental conditions. Because a large-scale application may incorporate a very large number of sensors in the field, however, it may not be possible or practical to periodically check the data integrity of each sensor on a manual basis. Furthermore, if the data integrity of a sensor is compromised, there may be no way to remedy the problem automatically. Thus, integrity of the data generated by sensors may present the highest level of vulnerability in the operation of an IoT system. For example, a data integrity issue in a single inexpensive sensor of a multi-million dollar IoT system can render the entire system unreliable, or in extreme cases, useless.

### SUMMARY

In one aspect, the disclosed technology relates to a system for calibrating a sensor communicatively coupled to a communications network. The system includes an emulator configured to, during operation, generate and provide to the sensor one or more inputs of known magnitude. The system also includes one or more computing devices communicatively coupled to the emulator and the sensor. At least one of the computing devices has stored therein data relating to response characteristics of the sensor. The one or more computing devices are configured to, during operation: cause the emulator to generate and provide to the sensor the one or more inputs of known magnitude; receive, via the communication network, one or more outputs of the sensor responsive to the one or more inputs of known magnitude; and generate calibration data for the sensor based on the one or more outputs of the sensor and the response characteristics of the sensor.

In another aspect of the disclosed technology, the one or more computing devices include a data gateway and a data management system.

In another aspect of the disclosed technology, the calibration data for the sensor incudes a calibration curve.

In another aspect of the disclosed technology, the one or more computing devices include a data base having the predetermined response characteristics of the sensor stored therein.

In another aspect of the disclosed technology, the system further includes a user interface communicatively coupled to at least one of the computing devices and configured to, during operation, permit a user to initiate the calibration of the sensor.

In another aspect of the disclosed technology, the user interface includes at least one of: a smart phone having a mobile application configured to permit the user to initiate the calibration of the sensor by way of the smart phone; and a desktop computer having a desktop application configured to permit the user to initiate the calibration of the sensor by way of the desktop computer.

In another aspect of the disclosed technology, the user interface is further configured to, during operation, display data and/or patterns of data acquired from the sensor.

In another aspect of the disclosed technology, the one or more computing devices are further configured to analyze data acquired from the sensor and recognize data patterns indicating a loss of data integrity in the sensor.

In another aspect of the disclosed technology, the one or more computing devices are further configured to initiate the calibration of the sensor in response to the loss of data integrity in the sensor.

In another aspect of the disclosed technology, the one or more computing devices are further configured to validate the results of the calibration.

In another aspect of the disclosed technology, the system further includes the sensor.

In another aspect of the disclosed technology, the communications network is the internet.

In another aspect, the disclosed technology relates to a method for automatically calibrating a sensor communicatively coupled to a communications network. The method includes providing an emulator configured to, during operation, generate and provide to the sensor one or more inputs of predetermined magnitude. The method also includes causing the emulator to generate and provide to the sensor the one or more inputs of predetermined magnitude; receiving, via the communication network, one or more outputs of the sensor responsive to the one or more inputs of predetermined magnitude; and generating calibration data for the sensor based on the one or more outputs of the sensor and the predetermined response characteristics of the sensor.

In another aspect of the disclosed technology, generating calibration data for the sensor based on the one or more outputs of the sensor and the predetermined response characteristics of the sensor characteristics of the sensor includes generating a calibration curve for the sensor.

In another aspect of the disclosed technology, the method further includes analyzing data acquired from the sensor and recognizing data patterns indicating a loss of data integrity in the sensor.

In another aspect of the disclosed technology, the method further includes initiating the calibration of the sensor in response to the loss of data integrity in the sensor.

In another aspect of the disclosed technology, the method further includes validating the results of the calibration.

In another aspect of the disclosed technology, validating the results of the calibration incudes: causing the emulator to generate and provide to the sensor one or more additional inputs of predetermined magnitude; receiving, via the communication network, one or more outputs of the sensor responsive to the one or more additional inputs of predetermined magnitude; and comparing the one or more additional inputs of predetermined magnitude to the one or more outputs of the sensor responsive to the one or more additional inputs of predetermined magnitude.

In another aspect of the disclosed technology, the method further includes providing a user interface, and initiating the calibration based on a manual input to the user interface.

In another aspect of the disclosed technology, the sensor is part of an internet of things system; and causing the emulator to generate and provide to the sensor the one or more inputs of predetermined magnitude includes causing the emulator to generate and provide to the sensor the one or more inputs of predetermined magnitude while the sensor is installed in the internet of things system.

### DESCRIPTION OF THE DRAWINGS

The following drawings are illustrative of particular embodiments of the present disclosure and do not limit the scope of the present disclosure. The drawings are not to scale and are intended for use in conjunction with the explanations provided herein. Embodiments of the resent disclosure will hereinafter be described in conjunction with the appended drawings.

FIG. 1 is a diagrammatic illustration of an embodiment of an automatic quality control and sensor calibration system 10.

FIG. 2 is a diagrammatic illustration of an IoT gateway of the system shown in FIG. 1.

FIG. 3 is a diagrammatic illustration of an embodiment of an emulator capable of use in system shown in FIG. 1, the emulator being for use in calibrating a vacuum sensor.

FIG. 4 is a diagrammatic illustration of another embodiment of an emulator capable of use in the system shown in FIG. 1, the emulator being for use in calibrating an analyzer for identifying machine failures.

FIG. 5 is an illustration of a screen display of a user interface of the system shown in FIG. 1, during calibration of the vacuum sensor shown in FIG. 3.

FIG. 6 is an illustration of a screen display of the user interface of the system shown in FIG. 1, during calibration of the analyzer shown in FIG. 4.

FIG. 7 is a block diagram depicting various functional aspects of the system shown in FIG. 1.

FIG. 8 is a flow diagram depicting operation of the system shown in FIG. 1 during calibration of a sensor by the system.

FIG. 9 is a front view of an IoT self-service temperature screening device.

FIG. 10 is a side view of the temperature screening device shown in FIG. 9.

FIG. 11 is a perspective view of a temperature sensor of the temperature screening device shown in FIGS. 9 and 10.

FIG. 12 is a table containing technical specifications for the temperature sensor shown in FIG. 11.

FIG. 13 is a side perspective view of a calibrator configured to perform an automated calibration of the temperature screening device shown in FIGS. 9 and 10.

FIG. 14 is a side perspective view of the temperature screening device and the calibrator shown in FIGS. 9, 10, and 13, depicting the calibrator snap-fitted onto the temperature screening device.

FIG. 15 is a diagrammatic illustration of the calibrator shown in FIGS. 13 and 14.

FIG. 16 depicts a login page and various other screen displays that guide a user through the initiation of a calibration process performed by the calibrator shown in FIGS. 13-15.

### DETAILED DESCRIPTION

The inventive concepts are described with reference to the attached figures, wherein like reference numerals represent like parts and assemblies throughout the several views. The figures are not drawn to scale and are provided merely to illustrate the instant inventive concepts. The figures do not limit the scope of the present disclosure or the appended claims. Several aspects of the inventive concepts are described below with reference to example applications for illustration. It should be understood that numerous specific details, relationships, and methods are set forth to provide a full understanding of the inventive concepts. One having ordinary skill in the relevant art, however, will readily recognize that the inventive concepts can be practiced without one or more of the specific details or with other methods. In other instances, well-known structures or operation are not shown in detail to avoid obscuring the inventive concepts.

An automatic quality control (QC) and sensor calibration system 10 is disclosed. The system 10 can be used to monitor, and if necessary, recalibrate in situ one or more sensors of an IoT system 100, or other types of systems that incorporate sensors. The system 10 incorporates an anomaly detection algorithm that can automatically detect, and determine the extent of, a loss or degradation of the integrity of the data produced by the sensors, which in turn may indicate a need to recalibrate the sensor. In addition, the system 10 can display to a user data and data patterns associated with one or more sensors, so that the user can identify anomalies that may indicate a loss of data integrity, including a loss of calibration; and a need for recalibration of the sensor. Upon the identification of a possible data-integrity issue with a sensor, the system 10 can initiate and perform an automatic field calibration of the sensor, and can validate the calibration results.

The term “sensor,” as used herein, encompasses, without limitation, devices configured to sense and transduce a physical parameter; and intelligent devices that incorporate such functionality. In some applications, for example, the transduced output of the sensor can be transmitted to a data gateway.

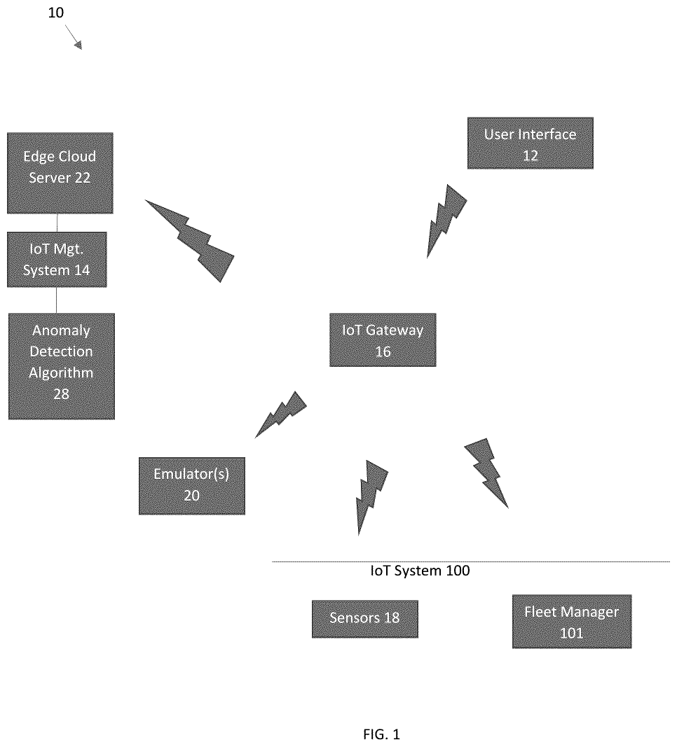

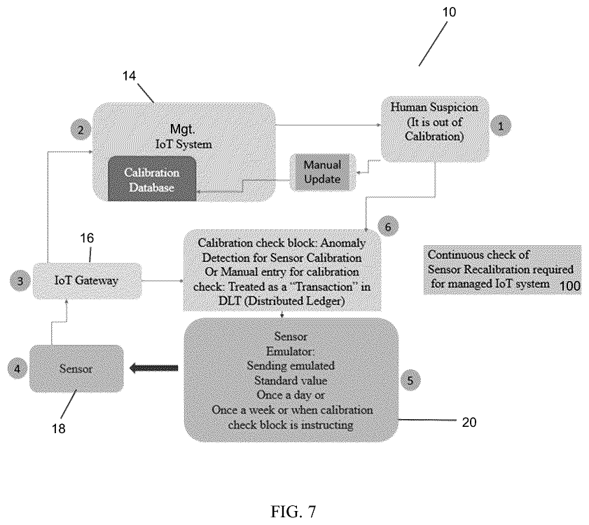

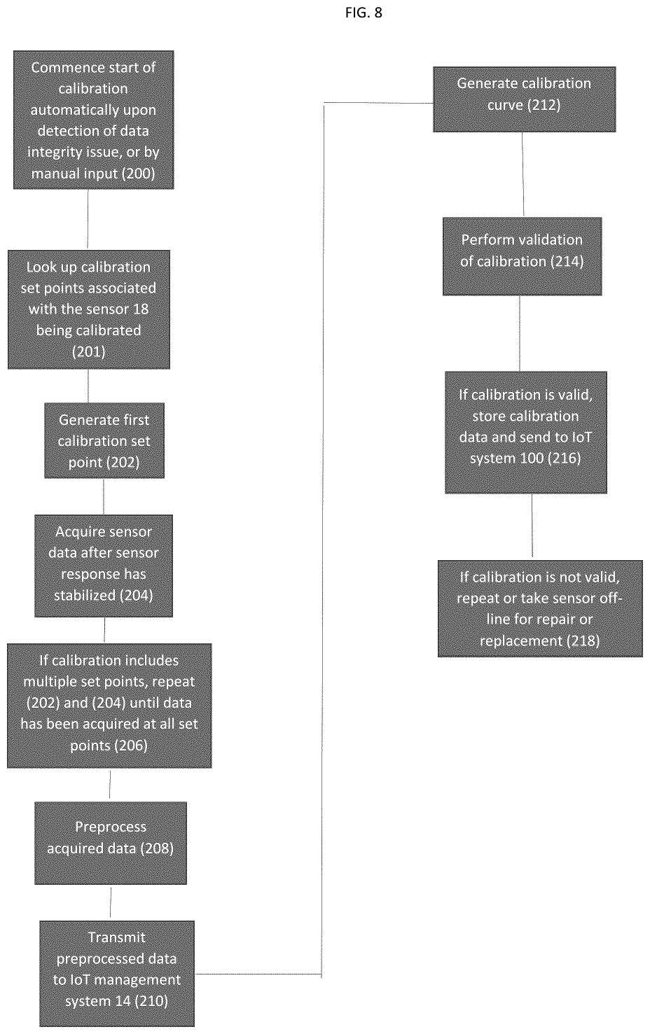

FIG. 1 is a diagrammatic illustration of the system 10. FIG. 7 depicts various functional aspects of the system 10, and FIG. 8 depicts the operation of the system 10 during calibration of a sensor. The system 10 comprises a user interface 12, and a data gateway in the form of an IoT gateway 16 communicatively coupled to the user interface 12. Also, the IoT gateway 16 can be communicatively coupled to a fleet manager 101 of the IoT system 100, so that the system 10 can share updated calibration data with the fleet manager 101, and provide the fleet manager 101 with the calibration status of the various sensors of the IoT system 100. The term “fleet manager,” as used herein, encompasses, without limitation, software applications that assist the users of IoT system in managing a fleet of devices such as remote sensors, controllers etc. Management functions performed by the fleet manager can include, without limitation, useful services such as registration and traceability of the devices for security purposes, remote firmware updates of the devices, remote diagnosis of the devices, calibration of the devices and system, etc.

The user interface 12 permits a user to monitor the status of the various sensors 18 that have been on-boarded onto, i.e., associated with, the system 10. The user interface 12 also permits the user to initiate a calibration process for a particular sensor 18. The user interface 12 can be any computing device or computing system that can display the data patterns and other information associated with the sensors 18; and that permits a user to enter inputs to the system 10, such as a command to initiate the calibration process for a particular sensor 18. For example, the user interface 12 can be a smart phone equipped with a suitable mobile application, or a desktop computer equipped with a suitable desktop application. The user interface 12 can communicate with the IoT gateway 16 by a suitable means such as, without limitation, Wi-Fi, a cellular network, a local area network, a wide area network, or a wired connection.

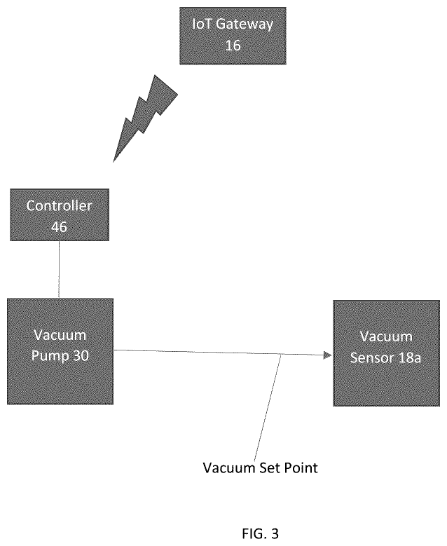

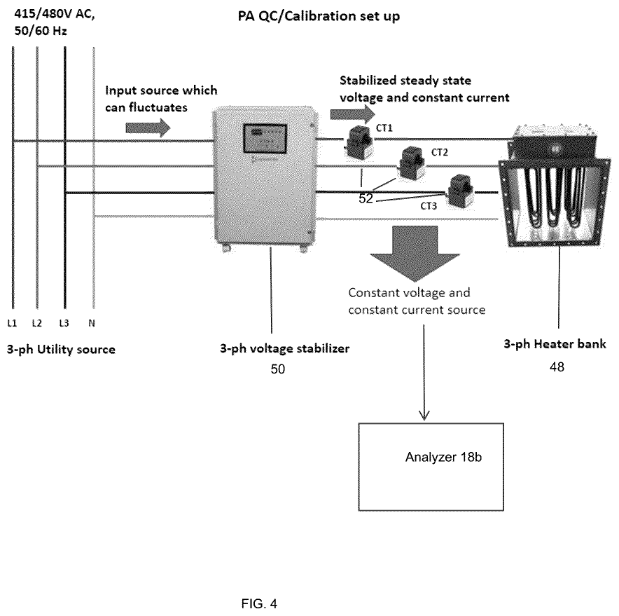

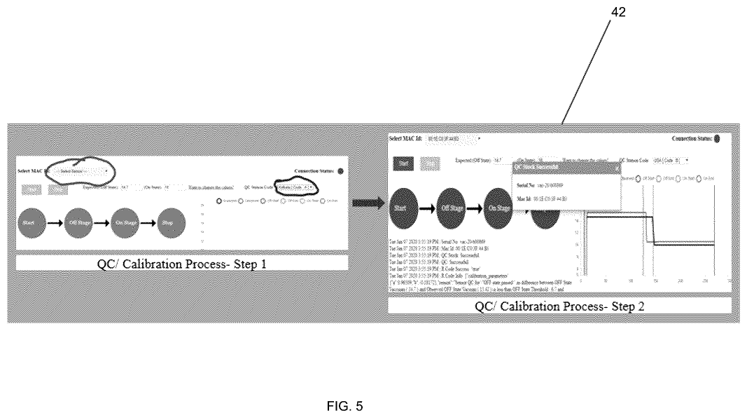

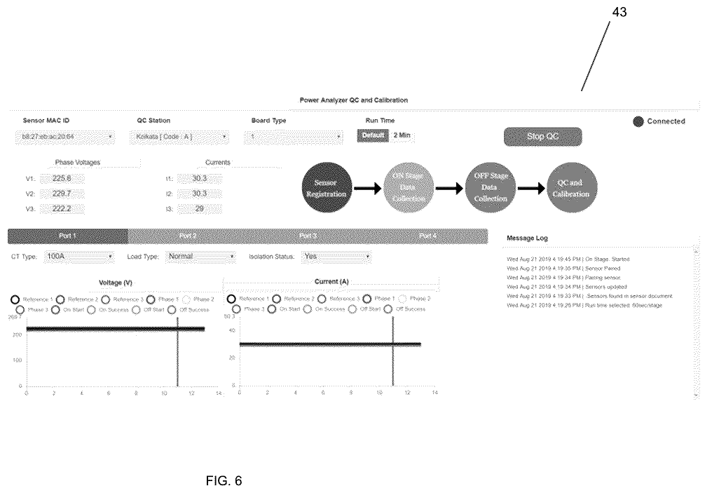

For example, FIG. 5 depicts the display 42 of a smart phone equipped with a mobile application that permits the smart phone to function as a user interface 12 for the system 10. The display 42 is depicted during the calibration of a vacuum sensor 44 shown in FIG. 3. As another example, FIG. 6 depicts the display 43 of a desktop computer equipped with a desktop application that permits the desktop computer to function as a user interface 12 for the system 10. The display 43 is depicted during the calibration of an analyzer 18b for detecting machinery failures, shown in FIG. 4.

The system 10 also includes a data management system in the form of an IoT management system 14, shown diagrammatically in FIGS. 1 and 7. The IoT management system 14 comprises a data base that includes a complete set of calibration data, including calibration curves and equations, for each on-boarded sensor 18. Each set of calibration data is mapped to a unique identifier, such as a MAC ID and a serial number, that associates the data with its corresponding sensor 18. In addition to the calibration data sets, the IoT management system 14 can include other information such as, without limitation, the calibration history for each sensor 18.

The IoT management system 14 can be incorporated into any suitable computing device including, without limitation, a cloud server or an edge-cloud server. In the illustrative embodiment disclosed herein, the IoT management system 14 is incorporated into an edge-cloud server 22, shown in FIG. 1. The edge-cloud server 22 is communicatively coupled to the IoT gateway 16; and can communicate with the IoT gateway 16 via a suitable means such as, without limitation, Wi-Fi, a cellular network, a local area network, a wide area network, or a wired connection.

A user can access and download the data processed by and stored in the IoT management system 14 through the user interface 12, via the communications link provided by the IoT gateway 16. For example, the user can obtain a visual representation of the calibration history for each on-boarded sensor 18 on the user interface 12. Also, the user interface 12 can provide a visual indication of data patterns generated by each sensor 18; and can display the results of statistical analyses performed on the sensor data to identify possible data-integrity issues. Also, updates and other changes to the calibration data stored in the IoT management system 14 and the IoT gateway 16 can be input via the user interface 12. In alternative embodiments, the edge-cloud server 22, or other computing device on which the IoT management system 14 is hosted, can be equipped with provisions that permit a user or data base manager to access, download, and update the data stored in the IoT management system 14 directly from the edge-cloud server 22.

The IoT management system 14 can include security provisions, such as blockchain based or similar DAG (direct acrylic graph) means, to protect against unauthorized database updates, e.g., tampering with the calibration data.

The IoT gateway 16 is communicatively coupled to the each of the sensors 18 by a suitable means such as, without limitation, Wi-Fi, a cellular network, a local area network, a wide area network, or a wired connection. In addition to facilitating communications between the various components of the system 10, the IoT gateway 16 is configured to execute the sensor calibration process, and a security check for the calibration.

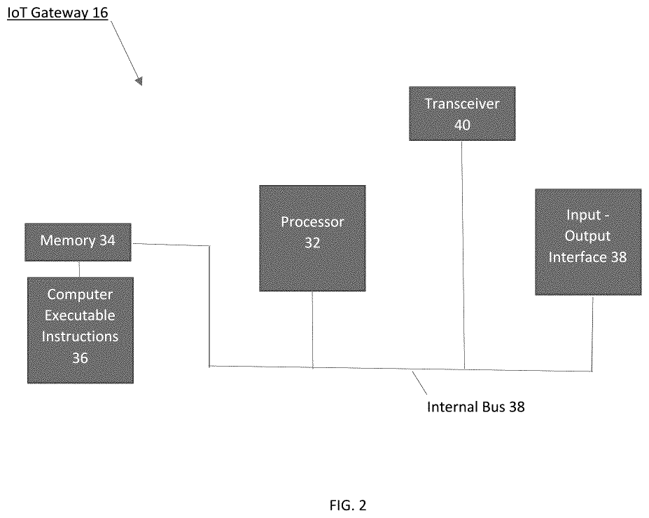

Referring the FIG. 2, the IoT gateway 16 comprises a processor 32. The processor 32 can be, for example, a microprocessor. The IoT gateway 16 also includes a memory 34, such as a random access memory, communicatively coupled to the processor 32; and computer executable instructions 36 stored on the memory 34. The processor 32 is configured so that the processor 32, upon executing the computer executable instructions 36, carries out the logical operations discussed below.

The IoT gateway 16 also comprises an internal bus 38 that facilitates communications between the various components of the IoT gateway 16; and an input-output interface 38 communicatively coupled to the processor 32. The IoT gateway 16 further includes a transceiver communicatively coupled to the input-output interface 38 and configured to facilitate wireless communications to and from the IoT gateway 16.

Specific details of the IoT gateway 16 are presented for illustrative purposes only. The IoT gateway 16 can have other configurations in alternative embodiments.

The system 10 further includes one or more emulators 20 that produce the reference inputs, or set points, required for calibration of the sensors 18. The emulators 20 are depicted diagrammatically in FIGS. 1 and 7.

Each emulator 20 is configured to provide a reference input corresponding to the specific type of sensor 18 undergoing calibration. For example, FIG. 3 depicts one particular embodiment of the emulator 20 in the form of a vacuum pump 44 configured to provide a vacuum. The vacuum is used as a reference input, or set point for the calibration of a vacuum sensor 18a. The vacuum sensor 18a can be used, for example, in an intelligent device for monitoring the health of conveying systems and predicting the failure or malfunction of pumps and blowers in the conveying systems.

The vacuum pump 30 is small, e.g., 0.5 horsepower; and is configured to produce a calibration set point of, for example, about ten pounds per square inch of vacuum. The vacuum pump 44 also can produce a second vacuum level of, for example, about 12 pounds per square inch, that can be used to validate the calibration process. The vacuum pump 44 can be controlled through a small, relay-based controller 46 communicatively coupled to the IoT gateway 16; the controller 46 can activate and deactivate the vacuum pump 44 in response to commands generated by and received from the IoT gateway 16.

The above description of the vacuum pump 44 as providing a single calibration reference point and a single validation reference point is presented for illustrative purposes only. The vacuum pump 44 can be configured to provide multiple calibration reference points and/or multiple validation reference points in alternative embodiments.

FIG. 4 depicts another example of a possible embodiment of the emulator 20 in the form of a three-phase, constant-load heater bank 48, a three-phase servo stabilizer 50, and three current transformers 52. This particular form of an emulator can be used to provide a steady, i.e., consistently varying, alternating current (AC) voltage. The voltage is used to calibrate an analyzer 18b that identifies machinery failures resulting from poor power quality and harmonic build ups.

Each phase of the servo stabilizer 50 is connected to a corresponding phase of the heater bank 48 via the primary winding of one of the current transformers 52. The servo stabilizer 50 generates a steady AC voltage with, for example, about 0.5% or less fluctuation between cycles; and the heater bank 48 draws a constant current from the servo stabilizer 50. The secondary winding of each current transformer 52 thus produces the steady AC voltage required to calibrate the analyzer 18b.

The system 10 is configured to analyze of the data acquired from the sensors 18 to identify possible data-integrity issues necessitating recalibration of the sensor 18. Upon the identification of a data-integrity issue with a particular senor 18, the recalibration process for that sensor 18 can be initiated manually by a user; or automatically by the IoT gateway 16.

The IoT management system 14 can include an anomaly detection algorithm 28 that, upon execution by the edge-cloud server 22, automatically detects data-integrity issues with the sensors 18. The anomaly detection algorithm 28 is depicted diagrammatically in FIG. 1. For example, the anomaly detection algorithm 28 can be configured to conduct periodic statistical analyses of the data acquired from each sensor 18. The statistical analyses can identify outliers in the acquired data for a particular sensor 18; and can determine when the quantity and magnitude of the outliers are indicative of a data-integrity issue requiring recalibration of the senor 18. Also, the statistical analyses can identify trends in the acquired data indicating a drift in the response of the sensor 18, or other conditions necessitating a recalibration. In addition, in some possible embodiments, the anomaly detection algorithm 28 can incorporate artificial intelligence based anomaly detection techniques to develop rules by which to evaluate data integrity for particular types of sensors.

A non-limiting example of an anomaly detection algorithm 28 is as follows. A plastic processing plant uses twenty vacuum pumps for a plastic conveying system. The health of each pump is monitored by a vacuum sensor mounted in the vacuum system proximate the pump, and vacuum data is extracted and sent to an on-site or public cloud in real time to assess the health of the pumps. Based on the assumptions that all of the vacuum pumps in the plant work with same routine, the age of the vacuum sensors during is roughly the same, and the vacuum sensors have experienced the same ambient conditions, anomalous sensor readings can be detected two ways.

First, during no-vacuum or ordinary rest condition, the vacuum sensors each should be giving the vacuum value expected at their particular altitude from sea level. Thus, all of the vacuum sensors should be sending approximately the same calibrated values during a no-vacuum or rest condition. If the output of any vacuum sensor deviates significantly from those of the rest of the group, the vacuum sensor with the anomalous output easily can be identified and isolated. Also, because all the vacuum pumps work with preset vacuum levels, the output values of the vacuum sensors can be compared to identify a vacuum sensor that has lost its calibration or otherwise is providing anomalous data, because the vacuum pattern histogram for that sensor will differ from those of the other vacuum sensors. Also, the vacuum histogram data for each sensor can be compared with the vacuum histogram for that sensor data at the time of its installation, as a baseline check for a loss of calibration or other anomalies.

In addition, the system 10 can be configured to perform a periodic validation process for each sensor 18, as referenced in FIG. 7. More specifically, the IoT gateway 16 can command the emulators 20 to send one or more set points to each sensor 18 on a predetermined periodic basis, e.g., daily, weekly, monthly, etc. The response of the sensor 18 can be transmitted to the edge-cloud server 22, where the anomaly detection algorithm 28 compares the response of the sensor 18 to the corresponding set point. The anomaly detection algorithm 28 interprets any differences between the sensor response and the set point that exceed a predetermined value as a data-integrity issue requiring recalibration.

The system 10 is configured to automatically initiate the calibration of a particular sensor 18 when one or more of the above-noted diagnostic checks indicate a need for recalibration of that senor 18. More specifically, once the anomaly detection algorithm 28 determines that a particular sensor 18 requires calibration, the anomaly detection algorithm 28 causes the edge-cloud server 22 to generate and issue a command that, when received by the IoT gateway 16, causes the IoT gateway 16 to initiate the calibration process for the particular sensor 18 referenced in the command by its unique identifier.

Alternatively, the system 10 can be configured to generate an alert to the user 18 when a diagnostic check indicates a need for recalibration of a senor 18. The alert can be displayed on the user interface 12, so that the user can determine whether, and when to initiate a calibration of the sensor 18 by way of a command input manually into the user interface 12.

The user can view data, data patterns, and calibration information for the sensors 18 via the user interface 12. Thus, in addition to the automated diagnostic checks noted above, the user can monitor and evaluate the status of the sensors 18; and the user can make an independent determination of whether a particular sensor 18 sensor needs to be recalibrated. For example, FIGS. 5 and 6 depict the display of data associated with the respective vacuum sensor 18a and analyzer 18b. A user can manually initiate the calibration process for a particular sensor 18 by selecting a virtual “Start” button that is displayed on the user interface 12. Once the “Start” button is touched by the user, the user interface 12 generates and issues a command that, when received by the IoT gateway 16, causes the IoT gateway 16 to initiate the calibration process for the particular sensor 18 referenced in the command by its unique identifier.

Once the calibration process has been initiated on a manual or automated basis (step 200 of FIG. 8), the calibration proceeds in a fully automated manner, i.e., without any action required on the part of the user. The calibration process is executed by the IoT gateway 16. The IoT gateway 16 initially queries the IoT management system 14 to look up the calibration set points associated with the sensor 18 being calibrated (step 201). In systems incorporating more than one emulator 20, the IoT gateway 16 also queries the IoT management system 14 to look up the particular emulator 20 associated with the sensor 18 being calibrated.

The IoT gateway 16 next issues a command that causes the appropriate emulator 20 to generate and apply to the sensor 18 a physical input corresponding to the first calibration set point (step 202). Once the output of the sensor 18 has stabilized, the IoT gateway 16 records the output (step 204). If the calibration data for the sensor 18 includes two or more set points, the above procedure is repeated until the response of the sensor 18 at each set point has been recorded (step 206).

The results of the calibration process, i.e., the recorded response of the sensor 18 at each calibration set point, are preprocessed locally by the IoT gateway 16 (step 208). More specifically, the IoT gateway 16 is configured to summarize and aggregate the calibration results, and to tactically analyze the results for deviations from the expected values, before the results are sent to the IoT management system 14 for further processing. Preprocessing of the data on the gateway 16 can substantially reduce the time and transmission cost of the calibration process, and can provide an added layer of security for the data transfer and the IoT network. For example, the loss in data integrity that can be identified by the IoT gateway 16 can be indicative of a data hack performed for fraudulent or otherwise malicious purposes. Thus, a data breach can be identified even under circumstances in which the hacking initiates a historical data pattern for spoofing or masking the hacking activities.

The preprocessed calibration data generated by the IoT gateway 16 is transmitted to the IoT management system 14 (step 210). The IoT management system 14 generates a new calibration curve for the sensor 18, based on the preprocessed calibration data, and the predetermined response characteristics of the sensor 18 stored in the IoT management system 14 (step 212).

The system 10 can be configured to validate the new calibration curve as follows. Once the calibration curve has been generated, the IoT management system 14 can issue a command, via the IoT gateway 16, that causes the emulator 20 to apply to the sensor 18 a physical input corresponding to a first validation set point. Once the output of the sensor 18 has stabilized, the IoT gateway 16 relays the output value to the IoT management system 14. If the validation data for the sensor 18 includes two or more validation set points, the above procedure is repeated until the response of the sensor 18 to each validation set point has been relayed to the IoT management system 14.

The IoT management system 14 compares the response of the sensor 18 at each validation set point with the validation set point itself. Agreement between the response and the validation set point within a predetermined margin is interpreted as an indication that the calibration is valid (step 214). Upon validation of the calibration, the IoT management system 14 stores the new calibration curve (step 216). Also, the IoT management system 14 causes the edge-cloud server 22 to transmit the new calibration curve to the fleet manager 101 of the IoT system 100 via the IoT gateway 16, so that the new calibration curve can used to process data subsequently acquired by the sensor 18 during normal operation of the IoT system 100.

If the validation process indicates that the calibration is not valid, i.e., if the response of the sensor 18 to each validation set point does not agree with the validation set point within the predetermined margin, the calibration can be repeated, and/or the sensor 18 can be taken off-line and repaired or replaced (step 218).

In alternative embodiments, the functionality of IoT management system 14 and the IoT gateway 16 can be integrated into a single computing device.

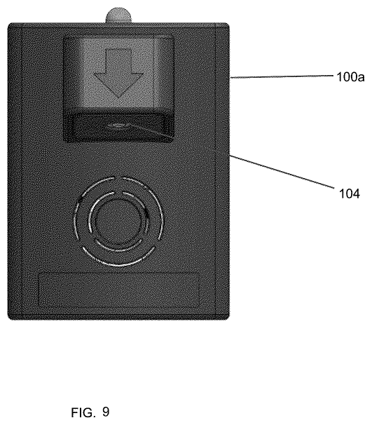

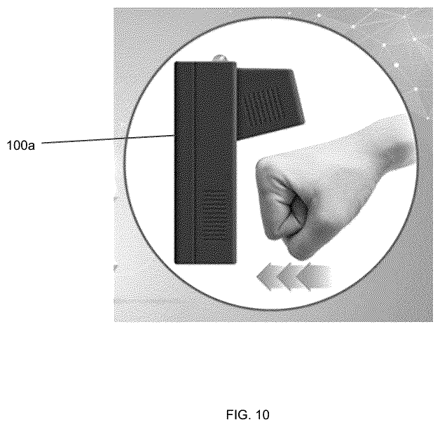

An example of the application of the automatic quality control (QC) and sensor calibration system 10 to a particular IoT system is described below. The particular IoT system is an IoT self-service temperature screening device 100a, depicted in FIGS. 9, 10, and 14. Because fever is the first symptom observed in many coronavirus patients, measurement of the body temperature of employees, students, customers, etc., has become a commonplace practice throughout the world. The temperature screening device 100a measures the skin temperature of the fist or wrist of the user to predict the user's core body temperature. The temperature screening device 100a works without any manual operator, and can be installed directly on a doorway, wall, or optional stand. An individual simply walks up to the temperature screening device 100a and places his or her fist or wrist area under the device 100a; and within one to two seconds the individual's temperature is taken, and a simple go/no-go instruction is issued via warning lights and/or a sound/buzzer system.

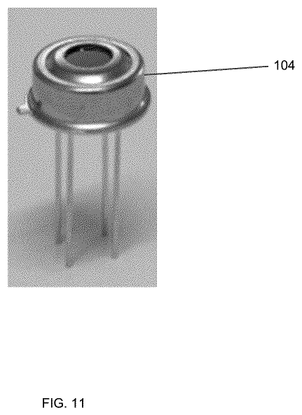

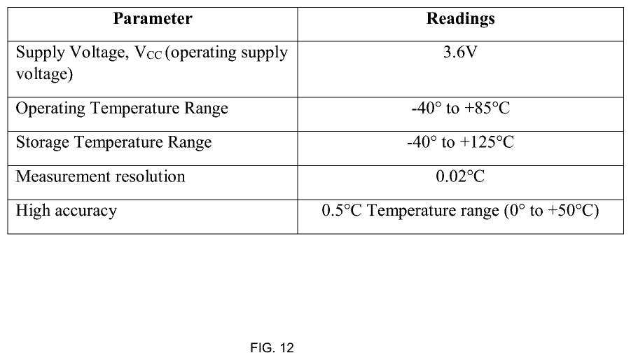

The temperature screening device 100a comprises a compact, infrared, non-contact temperature sensor 104 that measures human body temperature by detecting infrared light radiating from the fist or wrist area. The temperature sensor 104 is depicted in FIG. 11. The temperature sensor 104 is factory calibrated in wide temperature range, i.e., about −40° C. to about +125° C. for sensor temperature, and about −70° C. to about +380° C. for object temperature. The temperature sensor 104 operates at a voltage of about 3.3 VDC to about 5 VDC. Exemplary, on-limiting technical specifications for the temperature sensor 104 are presented in the table included as FIG. 12.

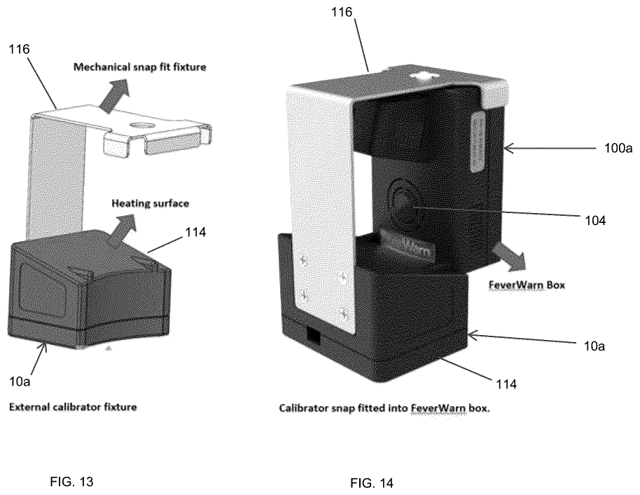

Although the temperature sensor 104 is factory calibrated, some data inaccuracies and loss of integrity may be observed after its use in diverse, and sometimes extreme environmental conditions. Because the temperature readings provided by the temperature screening device 100a may be used to screen for coronavirus and other deadly illnesses, it is important that the device 100a be checked and recalibrated on a regular basis. An easy to use, portable, and fully automated calibrator 10a for the temperature screening device 100a is depicted in FIGS. 13 and 14. The calibrator 10a is a specific application of the automatic QC and sensor calibration system 10 discussed generally above. Thus, unless stated otherwise, the above description of the system 10 applies equally to the calibrator 10a.

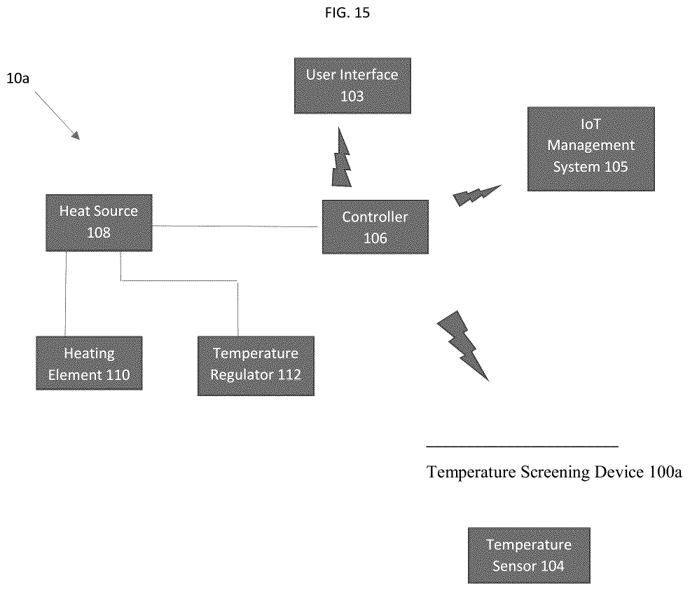

Referring to FIG. 15, a smart phone equipped with a mobile application can be employed as the user interface 103 for the calibrator 10a. A user can trigger an automated calibration process for the temperature sensor 104 by using the mobile application, after the user has onboarded the system 10a device via USB tethering, and after the calibrator 10a has been mounted on the temperature screening device 100a as depicted in FIG. 14.

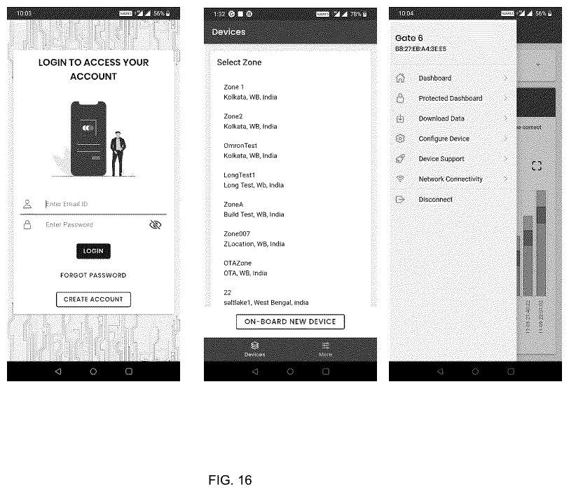

FIG. 16 depicts a login page and various other screen displays that guide the user through the initiation of the calibration process. After logging into the user's password-protected account, the user can select the specific temperature screening device 100a to be calibrated. The user then initiates the calibration by selecting the “Configure Device” tab on the left side menu, and then selecting the “Trigger Calibration” button and the “Auto Calibration” option that appear on the next two screen displays (not shown). Once the auto-calibration is started, there is no need to enter reference temperature manually, as the process is completely automated.

The user also has the option to display on the user interface 103 data, data patterns, and calibration data associated with the temperature sensor 104, as discussed above in relation to the user interface 12 of the system 10. Also, in alternative embodiments, the calibrator 10a can be configured to automatically conduct periodic validation checks of the data generated by the temperature sensor 104, as discussed above in relation to the system 10.

Referring to FIG. 15, the calibrator 10a further includes an IoT management system 105 that is substantially similar to the IoT management system 14 of the system 10. A Linux based, credit-card sized controller 106 with built-in RAM can be used as the IoT gateway for the calibrator 10a. The controller 106 can be Bluetooth and WiFi enabled, and can serve as a communications hub between the various components of the calibrator 10a, and the temperature screening device 100a. Also, the controller 106 executes the calibration process, and pre-processes the acquired data before sending the data to the IoT management system 105. The controller 106 also provides a layer of data security by providing additional data-integrity checks.

The IoT management system 105 can be hosted on an external computing device such as the edge-cloud server 22 referenced above in relation to the system 10. In alternative embodiments, the functionality of the IoT management system 105 can be integrated into the controller 106.

Referring to FIGS. 13-15, the calibrator 10a further comprises a black body heat source 108 that functions as an emulator for the calibration process. The black body heat source 108 comprises a heating element 110, and a precise feedback-controlled temperature regulator 112. The heat source 108 can operate on a micro-USB 5V input, and can communicate with the controller 106 via a universal asynchronous receiver-transmitter (UART). The heat source 108 and the controller 106 are mounted within a housing 114 made of material with high coefficient of thermal conductance and emissivity.

The calibrator 10a also includes a mounting bracket 116, shown in FIGS. 13 and 14. The mounting bracket 116 is attached to the housing 114, and can be snap-fitted onto the temperature screening device 100a so as to properly align the heat source 108 of the calibrator 10a with the temperature sensor 104 of the system 100a. As can be seen in FIGS. 13 and 14, the calibrator 10a is a portable and mechanically compact system that can be mounted easily on the system 100a.

One initiated, the calibration process for the temperature screening device 100a, including the entering of the calibration set points, is performed automatically. The calibration process can be performed in a room in which the ambient temperature is maintained between about 16° C. and about 35° C. The calibration is performed using two set points. The first and second set points can be hard coded into the controller 106. The controller 106 initiates the calibration process by triggering the heat source 108 to the first set point. The first set point can be, for example, about 36° C., which corresponds to the normal temperature of the human body. Once the black body surface of the heat source 108 has reached a steady-state temperature, the controller 106 acquires a temperature reading from the temperature sensor 104.

The controller 106 next triggers the heat source 108 to the second set point. The second set point can be, for example, about 40° C., which corresponds to an elevated human body temperature as can be experienced during a fever. Once the black body surface of the heat source 108 has reached a steady-state temperature, the controller 106 acquires another temperature reading from the temperature sensor 104.

The calibration data acquired from the temperature sensor 104 is pre-processed by the controller 106, as discussed above in relation to the system 10. The pre-processed data is sent to the IoT management system 105, which applies a two-point calibration algorithm to generate a calibration curve based on the newly acquired calibration data.

A validation process for the new calibration curve, similar to the validation process described above in relation to the system 10, can be performed by the IoT management system 105. If the calibration is found valid, the new calibration curve is stored in the IoT management system 105. Also, the new calibration curve is transmitted to the temperature screening device 100a via the controller 106, so that the new calibration curve can be used to process temperature readings acquired subsequently by the temperature sensor 104. Customers can be notified by, e-mail, SMS, or other suitable means once the entire process of calibration, validation, and data stockpiling has been completed.

If the calibration is deemed invalid, the calibration can be repeated, and/or the temperature screening device 100a can be taken off-line and repaired or replaced.

We claim:

1. A system for calibrating a sensor communicatively coupled to a communications network, the system comprising:
an emulator configured to, during operation, generate and provide to the sensor one or more inputs of known magnitude; and
one or more computing devices communicatively coupled to the emulator and the sensor, the one or more computing devices having stored therein data relating to response characteristics of the sensor, wherein the one or more computing devices each comprise a processor, a memory communicatively coupled to the processor, and computer executable instructions stored on the memory, and are configured to, during operation:
analyze data acquired from the sensor and recognize data patterns indicating a loss of data integrity in the sensor;
initiate a calibration of the sensor in response to the loss of data integrity in the sensor by:
causing the emulator to generate and provide to the sensor the one or more inputs of known magnitude;
receiving, via the communication network, one or more outputs of the sensor responsive to the one or more inputs of known magnitude; and
generating calibration data for the sensor based on the one or more outputs of the sensor and the response characteristics of the sensor.

2. The system of claim 1, wherein the one or more computing devices comprise a data gateway and a data management system.

3. The system of claim 1, wherein the calibration data for the sensor comprises a calibration curve.

4. The system of claim 1, wherein the one or more computing devices comprise a data base having the predetermined response characteristics of the sensor stored therein.

5. The system of claim 1, further comprising a user interface communicatively coupled to at least one of the computing devices and configured to, during operation, permit a user to initiate the calibration of the sensor.

6. The system of claim 5, wherein the user interface comprises at least one of:
a smart phone comprising a mobile application configured to permit the user to initiate the calibration of the sensor by way of the smart phone; and
a desktop computer comprising a desktop application configured to permit the user to initiate the calibration of the sensor by way of the desktop computer.

7. The system of claim 5, wherein the user interface is further configured to, during operation, display data and/or patterns of data acquired from the sensor.

8. The system of claim 1, wherein the one or more computing devices are further configured to validate the results of the calibration.

9. The system of claim 1, further comprising the sensor.

10. The system of claim 1, wherein the communications network is the internet.

11. A method for automatically calibrating a sensor communicatively coupled to a communications network, the method comprising:
providing an emulator configured to, during operation, generate and provide to the sensor one or more inputs of predetermined magnitude; and
causing the emulator to generate and provide to the sensor the one or more inputs of predetermined magnitude;
analyzing data acquired from the sensor and recognizing data patterns indicating a loss of data integrity in the sensor; and
initiating a calibration of the sensor in response to the loss of data integrity in the sensor by:
receiving, via the communication network, one or more outputs of the sensor responsive to the one or more inputs of predetermined magnitude; and
generating calibration data for the sensor based on the one or more outputs of the sensor and the predetermined response characteristics of the sensor.

12. The method of claim 11, wherein generating calibration data for the sensor based on the one or more outputs of the sensor and the predetermined response characteristics of the sensor characteristics of the sensor comprises generating a calibration curve for the sensor.

13. The method of claim 11, further comprising validating the results of the calibration.

14. The method of claim 13, wherein validating the results of the calibration comprises:
causing the emulator to generate and provide to the sensor one or more additional inputs of predetermined magnitude;
receiving, via the communication network, one or more outputs of the sensor responsive to the one or more additional inputs of predetermined magnitude; and
comparing the one or more additional inputs of predetermined magnitude to the one or more outputs of the sensor responsive to the one or more additional inputs of predetermined magnitude.

15. The method of claim 11, further comprising providing a user interface, and initiating the calibration based on a manual input to the user interface.

16. The method of claim 11, wherein:
the sensor is part of an internet of things system; and
causing the emulator to generate and provide to the sensor the one or more inputs of predetermined magnitude comprises causing the emulator to generate and provide to the sensor the one or more inputs of predetermined magnitude while the sensor is installed in the internet of things system.
